(19) United States
(12) Patent Application Publication
(10) Pub. No.: US 2010/0105666 A1
(43) Pub. Date: Apr. 29, 2010

(54) TRIAZABENZO (A)NAPHTHO(2,1,8-CDE) AZULENE COMPOUNDS.

(76) Inventors: Jean-Daniel Brion, Saint Leu La Foret (FR); Maud Hervet, Sceaux (FR); Anne Moreau, Lannion (FR); Zafiarisoa Dolor Renko, Les Ulis (FR); Alain Le Ridant, Neuilly Sur Sein (FR); Catherine Harpey, Paris (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 12/448,627

(22) PCT Filed: Jan. 4, 2008
(86) PCT No.: PCT/FR2008/000012

§ 371 Date: Dec. 10, 2009

(30) Foreign Application Priority Data
Jan. 5, 2007 (FR) 0700046

(51) Int. Cl. A61K 31/551; C07D 487/22; A61P 25/22; A61P 25/16; A61P 25/24

(52) U.S. Cl. 514/219; 540/555; 540/492

(57) ABSTRACT

Compounds of formula (I):

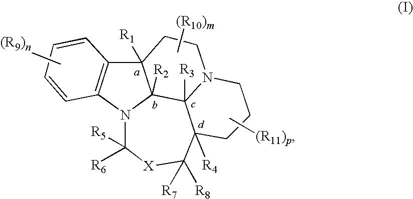

wherein:
R₁ represents hydrogen, (C₁-C₆)alkyl, (C₁-C₆)aminoalkyl or (C₁-C₆)hydroxyalkyl,
R₂ represents hydrogen,
or R₁ and R₂, together with the carbon atoms carrying them, form a carbon-carbon bond,
R₃ represents hydrogen,
R₄ represents hydrogen, methyl or (C₃-C₆)alkyl, (C₁-C₆)aminoalkyl, (C₁-C₆)hydroxyalkyl, aryl-(C₁-C₆)alkyl or heterocycloalkyl-(C₁-C₆)alkyl,
or R₃ and R₄ together with the carbon atoms carrying them, form a carbon-carbon bond,
R₅, R₆, R₇ and R₈ represent hydrogen,
or a pair of geminal substituents (R₅ and R₆ and/or R₇ and R₈) form an oxo, thioxo or imino group,
R₉ represents hydrogen, halogen, (C₁-C₆)alkyl, (C₁-C₆)alkoxy, hydroxy, cyano, nitro, (C₁-C₆)polyhaloalkyl or optionally substituted amino,
R₁₀ and R₁₁ represent hydrogen, halogen, (C₁-C₆)alkyl, (C₁-C₆)alkoxy, hydroxy, cyano, nitro, (C₁-C₆)polyhaloalkyl or optionally substituted amino,
n represents an integer between 0 and 4 inclusive,
m represents an integer between 0 and 2 inclusive,
p represents an integer between 0 and 3 inclusive,
X represents a group NR₁₂,
R₁₂ represents hydrogen, (C₁-C₆)alkyl, (C₂-C₆)alkenyl, aryl-(C₁-C₆)alkyl or (C₁-C₆)polyhaloalkyl,
   their enantiomers, diastereoisomers and N-oxides, and also addition salts thereof with a pharmaceutically acceptable acid or base.

The present invention relates to new triazabenzo[a]naphtho[2,1,8-cde]azulene compounds, to a process for their preparation and to pharmaceutical compositions containing them.

The literature provides numerous examples of compounds exhibiting an eburnane structure, this being the case especially with the patent specification U.S. Pat. No. 3,454,583, which deals with vincamine (methyl (3α,14β,16α)-(14,15-dihydro-14-hydroxy-eburnamenine-14-carboxylate) and compounds thereof with regard to their vasodilatory properties. The Patent Applications FR 2 433 528 and FR 2 381 048 present new 20,21-dinoreburn-amenine compounds and the Patent Application EP 0 287 468 presents new 17-aza-20,21-dinoreburnamenine compounds. The Patent Application EP 0 658 557 describes eburnane compounds modified in the 14- and 15-positions of the eburnane skeleton. The Patent Application EP 0 563 916 describes 1H-indole-cyclohexanecarboxamide compounds.

Besides the fact that they are new, the compounds of the present invention have very valuable pharmacological properties. In particular, they have been found to be powerful selective or non-selective tyrosine hydroxylase inducers.

More specifically, the present invention relates to compounds of formula (I):

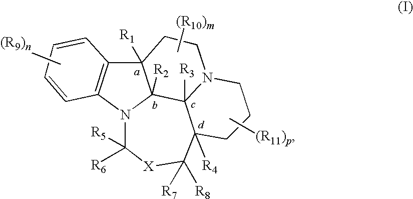

wherein:
R₁, represents a hydrogen atom or a linear or branched (C₁-C₆)alkyl group, a linear or branched (C₁-C₆)aminoalkyl group or a linear or branched (C₁-C₆)hydroxyalkyl group,
R₂ represents a hydrogen atom,
or R₁ and R₂, together with the carbon atoms carrying them, form a carbon-carbon bond,
R₃ represents a hydrogen atom,
R₄ represents a hydrogen atom or a methyl or linear or branched (C₃-C₆)alkyl group, a linear or branched (C₁-C₆)aminoalkyl group, a linear or branched (C₁-C₆)hydroxyalkyl group, an aryl-(C₁-C₆)alkyl group in which the alkyl moiety is linear or branched, or a heterocycloalkyl-(C₁-C₆)alkyl group in which the alkyl moiety is linear or branched,
or R₃ and R₄, together with the carbon atoms carrying them, form a carbon-carbon bond,
R₅, R₆, R₇ and R₈, which may be identical or different, represent, each independently of the others, a hydrogen atom
or a pair of geminal substituents (R₅ and R₆ and/or R₇ and R₈) form an oxo, thioxo or imino group,
R₉ represents a hydrogen or halogen atom or an optionally substituted, linear or branched (C₁-C₆)alkyl group, a linear or branched (C₁-C₆)alkoxy group, a hydroxy group, a cyano group, a nitro group, a linear or branched (C₁-C₆)polyhaloalkyl group or an amino group (optionally substituted by one or two linear or branched (C₁-C₆)alkyl(s), linear or branched (C₂-C₆)alkenyl(s), it being possible for the alkyls and alkenyls to be identical or different),
R₁₀ and R₁₁, which may be identical or different, represent, each independently of the other, a hydrogen or halogen atom or a linear or branched (C₁-C₆)alkyl group, a linear or branched (C₁-C₆)alkoxy group, a hydroxy group, a cyano group, a nitro group, a linear or branched (C₁-C₆)polyhaloalkyl group or an amino group (optionally substituted by one or two linear or branched (C₁-C₆)alkyl(s), linear or branched (C₂-C₆)alkenyl(s), it being possible for the alkyls and alkenyls to be identical or different),
n represents an integer between 0 and 4 inclusive (0, 1, 2, 3 or 4),
m represents an integer between 0 and 2 inclusive (0, 1 or 2),
p represents an integer between 0 and 3 inclusive (0, 1, 2 or 3),
X represents a group NR₁₂,
R₁₂ represents a hydrogen atom or an optionally substituted, linear or branched (C₁-C₆)alkyl group, an optionally substituted, linear or branched (C₂-C₆)alkenyl group, an aryl-(C₁-C₆)alkyl group in which the alkyl moiety is linear or branched, or a linear or branched (C₁-C₆)polyhaloalkyl group,
   to their enantiomers, diastereoisomers, N-oxides, and also to addition salts thereof with a pharmaceutically acceptable acid or base,
   it being understood that:
   arylalkyl means an aryl-alkyl group in which the alkyl group denotes a linear or branched, chain of 1 to 6 carbon atoms and the aryl group denotes an optionally substituted phenyl or naphthyl group,
   the expression “optionally substituted” when referring to linear or branched (C₁-C₆)alkyl, linear or branched (C₂-C₆)alkenyl or arylalkyl groups means that these groups may be substituted by one or more halogen atoms, by one or more groups hydroxy, linear or branched (C₁-C₆)alkoxy or amino (optionally substituted by one or two identical or different, linear or branched (C₁-C₆)alkyl groups),
   a, b, c and d denote the chiral centres which may possibly be present in the compounds of formula (I).

Among the pharmaceutically acceptable acids there may be mentioned, without implying any limitation, hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine, lysine etc.

An advantageous embodiment relates to compounds of formula (I) wherein X represents a group NR₁₂ wherein R₁₂ represents a hydrogen atom.

Another advantageous embodiment relates to compounds of formula (I) wherein R₁, R₂, R₃ and R₄ each represent a hydrogen atom.

Another particular embodiment of the invention relates to compounds of formula (I) wherein R₁ and R₂, together with the carbon atoms carrying them, form a carbon-carbon bond.

R₉ advantageously represents a hydrogen or halogen atom.

R₁₀ and R₁₁ advantageously each represent a hydrogen atom.

Preferred compounds of the invention are compounds of formula (I) wherein each pair of geminal substituents (R₅ and R₆, and R₇ and R₈) form an oxo group.

Another particular embodiment of the invention relates to compounds of formula (I) wherein R₅ and R₆ each represent a hydrogen atom and the pair of geminal substituents (R₇ and R₈) form an oxo group.

Even more especially, the invention relates to compounds of formula (I) which are:
(5aRS,12aSR,12bSR,12cSR)-7-chloro-2,3,4,5,5a,10,11,12a,12b,12c-decahydro-1H,12H-3a,9b,11-triazabenzo[a]naphtho[2,1,8-cde]azulen-12-one,
(5aRS,12aSR,12bSR,12cSR)-7-chloro-2,3,5a,12a,12b,12c-hexahydro-1H,4H-3a,9b,11-triazabenzo[a]naphtho[2,1,8-cde]azulene-10,12(5H,11H)-dione,
(12aRS,12bRS)-7-chloro-2,3,12a,12b-tetrahydro-1H,4H-3a,9b,11-triazabenzo[a]naphtho[2,1,8-cde]azulene-10,12(5H,11H)-dione,
(5aRS,12aSR,12bSR,12cSR)-2,3,5a,12a,12b,12c-hexahydro-1H,4H-3a,9b,11-triazabenzo[a]naphtho[2,1,8-cde]azulene-10,12(5H,11H)-dione,
(5aS,12aR,12bR,12cR)— or (5aR,12aS,12bS,12cS)-2,3,5a,12a,12b,12c-hexahydro-1H,4H-3a,9b,11-triazabenzo[a]naphtho[2,1,8-cde]azulene-10,12(5H,11H)-dione (enantiomer β),
(5aR,12aS,12bS,12cS)— or (5aS,12aR,12bR,12cR)-2,3,5a,12a,12b,12c-hexahydro-1H,4H-3a,9b,11-triazabenzo[a]naphtho[2,1,8-cde]azulene-10,12(5H,11H)-dione (enantiomer β),
(12aRS,12bRS)-2,3,12a,12b-tetrahydro-1H,4H-3a,9b,11-triazabenzo[a]naphtho[2,1,8-cde]azulene-10,12(5H,11H)-dione.

The enantiomers, diastereoisomers and N-oxides of the preferred compounds, and also addition salts thereof with a pharmaceutically acceptable acid or base are an integral part of the invention.

The notation (5aRS,12aSR,12bSR,12cSR)— followed by the name of the compound means that the product obtained is a racemic mixture and that therefore both configurations are possible.

By way of example:

(5aRS,12aSR,12bSR,12cSR)-7-chloro-2,3,4,5,5a,10,11,12a,12b,12c-decahydro-1H,12H-3a,9b,11-triazabenzo[a]naphtho[2,1,8-cde]azulen-12-one means that the product obtained, the racemic mixture, contains (5aR,12aS,12bS,12cS)-7-chloro-2,3,4,5,5a,10,11,12a,12b,12c-decahydro-1H,12H-3a,9b,11-triazabenzo[a]naphtho[2,1,8-cde]azulen-12-one and (5aS,12aR,12bR,12cR)-7-chloro-2,3,4,5,5a,10,11,12a,12b,12c-decahydro-1H,12H-3a,9b,11-triazabenzo[a]naphtho[2,1,8-cde]azulen-12-one.

The notation (5aR,12aS,12bS,12cS)— or (5aS,12aR,12bR,12cR)— followed by the name of the compound means that the product obtained is an optically pure enantiomer.

By way of example:

(5aS,12aR,12bR,12cR)— or (5aR,12aS,12bS,12cS)-7-chloro-2,3,4,5,5a,10,11,12a,12b,12c-decahydro-1H,12H-3a,9b,11-triazabenzo[a]naphtho[2,1,8-cde]azulen-12-one means that the product obtained, the optically pure enantiomer, is (5aS,12aR,12bR,12cR)-7-chloro-2,3,4,5,5a,10,11,12a,12b,12c-decahydro-1H,12H-3a,9b,11-triazabenzo[a]naphtho[2,1,8-cde]azulen-12-one or (5aR,12aS,12bS,12cS)-7-chloro-2,3,4,5,5a,10,11,12a,12b,12c-decahydro-1H,12H-3a,9b,11-triazabenzo[a]naphtho[2,1,8-cde]azulen-12-one.

Enantiomer α and enantiomer β mean the optically pure enantiomers of the corresponding racemic mixture.

By way of example:

(5aR,12aS,12bS,12cS)— or (5aS,12aR,12bR,12cR)-7-chloro-2,3,4,5,5a,10,11,12a,12b,12c-decahydro-1H,12H-3a,9b,11-triazabenzo[a]naphtho[2,1,8-cde]azulen-12-one (enantiomer α) means that if enantiomer α represents (5aR,12aS,12bS,12cS)-7-chloro-2,3,4,5,5a,10,11,12a,12b,12c-decahydro-1H,12H-3a,9b,11-triazabenzo[a]naphtho[2,1,8-cde]azulen-12-one then enantiomer 13 represents (5aS,12aR,12bR,12cR)-7-chloro-2,3,4,5,5a,10,11,12a,12b,12c-decahydro-1H,12H-3a,9b,11-triazabenzo[a]naphtho[2,1,8-cde]azulen-12-one.

The present invention relates also to a process for the preparation of compounds of formula (I), which process is characterised in that there is used as starting material a compound of formula (II) (the synthesis route of which is described by R. N. Schut and T. J. Leipzig, J. Het. Chem. 3, (1966), 101-102):

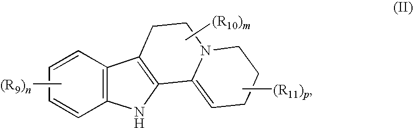

wherein R₉, R₁₀, R₁₁, n, m and p are as defined for formula (I),
which is subjected to the action of a compound of formula (III):

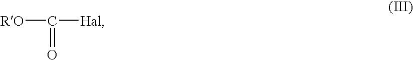

wherein R′ represents a linear or branched (C₁-C₆)alkyl group and Hal represents a halogen atom, to yield the compound of formula (IV):

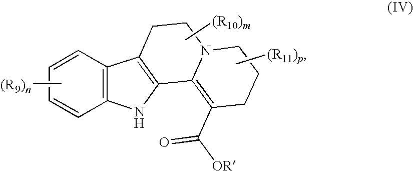

wherein R′, R₉, R₁₀, R₁₁, n, m and p are as defined hereinbefore,
which is subjected to a hydrogenation reaction in the presence of sodium cyanoborohydride and acetic acid in an anhydrous medium to yield a mixture of cis enantiomers of formula (Vcis):

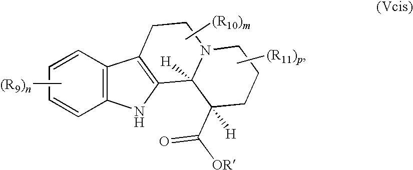

wherein R′, R₉, R₁₀, R₁₁, n, m and p are as defined hereinbefore,
which is subjected to an epimerisation reaction in the presence of an alkali metal hydride in an anhydrous solvent at 0° C. to yield the mixture of trans enantiomers of formula (Vtrans):

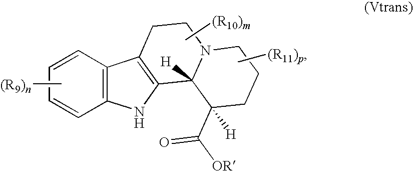

wherein R′, R₉, R₁₀, R₁₁, n, m and p are as defined hereinbefore,
the totality of which compounds of formulae (Vcis) and (V trans) form the totality of compounds of formula (V):

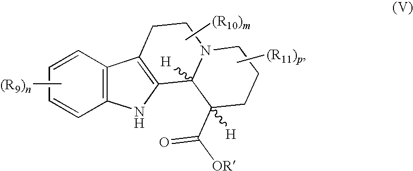

wherein R′, R₉, R₁₀, R₁₁, n, m and p are as defined hereinbefore,
which compounds of formula (V) are subjected to a hydrogenation reaction in the presence of sodium cyanoborohydride and trifluoroacetic acid to yield the compound of formula (VI):

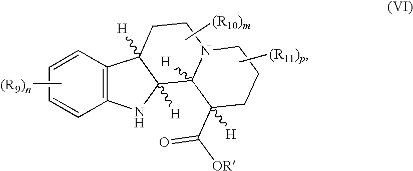

wherein R′, R₉, R₁₀, R₁₁, n, m and p are as defined hereinbefore,
which is subjected:
either to the action of ammonium chloride in the presence of trimethylaluminium in an anhydrous solvent at 0° C. to yield the compound of formula (VII):

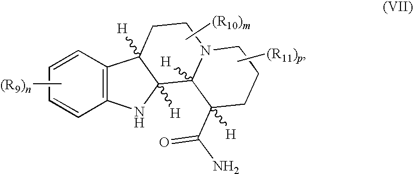

wherein R₉, R₁₀, R₁₁, n, m and p are as defined hereinbefore,
which is cyclised in the presence of paraformaldehyde and acetic acid to yield the compound of formula (I/a), a particular case of the compounds of formula (I):

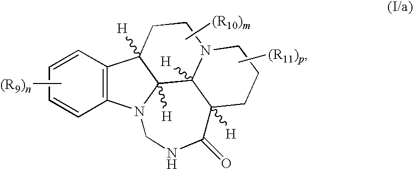

wherein R₉, R₁₀, R₁₁, n, m and p are as defined hereinbefore,
or to the action of potassium cyanate in the presence of trifluoroacetic acid in an anhydrous solvent to yield the compound of formula (VIII):

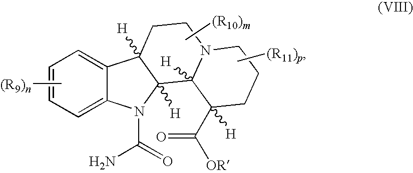

wherein R′, R₉, R₁₀, R₁₁, n, m and p are as defined hereinbefore,
which is subjected to the action of potassium carbonate to yield the compound of formula (I/b), a particular case of the compounds of formula (I):

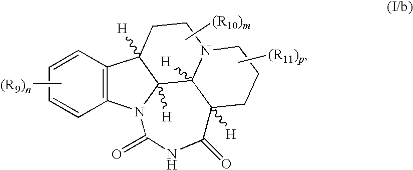

wherein R₉, R₁₀, R₁₁, n, m and p are as defined hereinbefore,
the compounds of formulae (I/a) and (I/b) forming the totality of the compounds of formula (I/c):

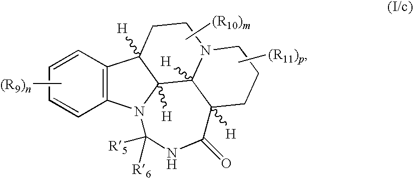

wherein R′₅ and R′₆ each represent a hydrogen atom or together form an oxo group, and R₉, R₁₀, R₁₁, n, m and p are as defined hereinbefore,
which is reduced in the presence of an alkali metal hydride to yield the compound of formula (I/d), a particular case of the compounds of formula (I):

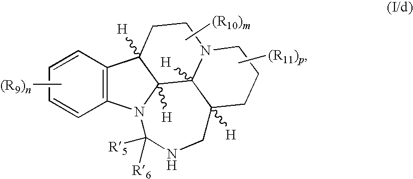

wherein R′₅, R′₆, R₉, R₁₀, R₁₁, n, m and p are as defined hereinbefore,
which compounds of formulae (I/c) and (I/d) are subjected to the action of a compound of formula (IX):

R′₁₂-Hal (IX),

wherein R′₁₂ is as defined for R₁₂ with the exception of hydrogen and Hal represents a halogen atom, to yield the compound of formula (I/e), a particular case of the compounds of formula (I):

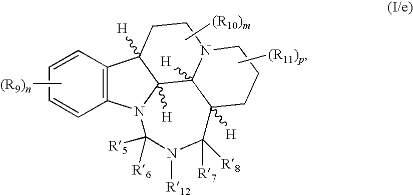

wherein R′₇ and R′₈ each represent a hydrogen atom or together form an oxo group and R′₅, R′₆, R₉, R₁₀, R₁₁, n, m and p are as defined hereinbefore,
which is subjected, when R′₅ and R′₆ and/or R′₇ and R′₈ together form an oxo group, to the action of Lawesson's reagent to yield a compound of formula (I/f₁), (I/f₂) or (I/f₃), a particular case of the compounds of formula (I):

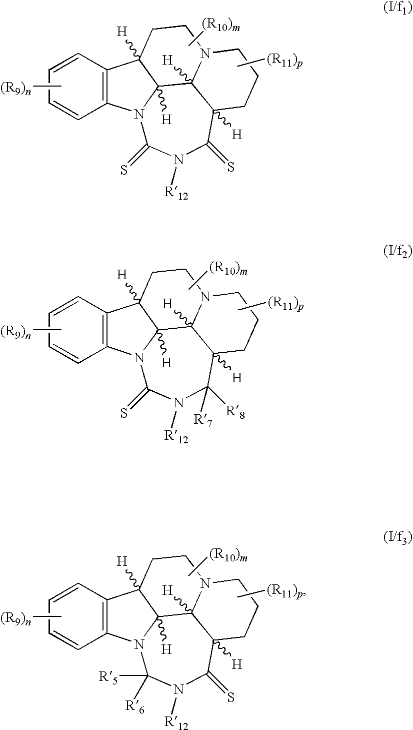

wherein R′₅, R′₆, R′₇, R′₈, R₉, R₁₀, R′₁₂, n, m and p are as defined hereinbefore,
the compounds of formulae (I/a) to (I/f₃) forming the totality of the compounds of formula (I), which may be purified according to a conventional separation technique, may be converted, if desired, into their addition salts with a pharmaceutically acceptable acid or base and may be separated, where appropriate, into their isomers according to a conventional separation technique.

The compounds of formulae (II), (III) and (IX) are, either commercially available or obtained by conventional reactions of organic synthesis well known to the person skilled in the art.

The compounds of formula (I) have valuable pharmacological properties, especially that of being powerful tyrosine hydroxylase (TH) inducers. It is known that tyrosine hydroxylase is a rate-limiting enzyme which controls particularly the synthesis of neurotransmitters in central catecholaminergic and dopaminergic neurons. The rate of synthesis of those neurotransmitters is related especially to the appearance of tonic brain dysfunctions constituting numerous behavioural pathologies in humans, such as anxiety, psychoses, depression, stress etc.

By virtue of their ability to induce tyrosine hydroxylase, the compounds of the invention will accordingly be used therapeutically in the treatment of depression, anxiety, disorders of memory in the course of ageing and/or neurodegenerative diseases, and in the palliative treatment of Parkinson's disease, and for adaptation to stress.

The present invention relates also to pharmaceutical compositions comprising, as active ingredient, at least one compound of formula (I), its enantiomers, diastereoisomers, N-oxides, or one of its addition salts with a pharmaceutically acceptable acid or base, alone or in combination with one or more pharmaceutically acceptable, inert, non-toxic excipients or carriers.

The pharmaceutical compositions thereby obtained will generally be presented in a dosage form; for example, they may take the form of tablets, dragées, capsules, suppositories, injectable or drinkable solutions and may be administered by the oral, rectal, intramuscular or parenteral route.

Among the pharmaceutical compositions according to the invention there may be mentioned more especially those that are suitable for oral, parenteral (intravenous, intramuscular or subcutaneous), per- or trans-cutaneous, intravaginal, rectal, nasal, perlingual, buccal, ocular or respiratory administration.

The pharmaceutical compositions according to the invention for parenteral injections especially include aqueous and non-aqueous sterile solutions, dispersions, suspensions or emulsions as well as sterile powders for the reconstitution of injectable solutions or dispersions.

The pharmaceutical compositions according to the invention for solid oral administration especially include tablets or dragées, sublingual tablets, sachets, capsules and granules, and for liquid oral, nasal, buccal or ocular administration especially include emulsions, solutions, suspensions, drops, syrups and aerosols.

The pharmaceutical compositions for rectal or vaginal administration are preferably suppositories or ovules, and those for per- or trans-cutaneous administration especially include powders, aerosols, creams, ointments, gels and patches.

The above-mentioned pharmaceutical compositions illustrate the invention but do not limit it in any way.

Among the pharmaceutically acceptable, inert, non-toxic acceptable excipients or carriers there may be mentioned, by way of example and without implying any limitation, diluents, solvents, preservatives, wetting agents, emulsifiers, dispersants, binders, swelling agents, disintegrants, retardants, lubricants, absorbency agents, suspension agents, colourants, flavourings etc.

The useful dosage varies according to the age and weight of the patient, the route of administration, the pharmaceutical composition used, the nature and severity of the disorder, and whether any associated treatments are being taken. The dosage ranges from 0.1 mg to 100 mg per day in one or more administrations.

The following Examples illustrate the invention but do not limit it in any way.

The starting materials used are known products or are prepared according to known procedures. The various Preparations yield synthesis intermediates that are useful in preparation of compounds of the invention.

The structures of the compounds described in the Examples and in the Preparations were determined in accordance with the usual spectrometric techniques (infrared, nuclear magnetic resonance, mass spectrometry etc.).

The melting points were determined using a TOTTOLI apparatus (without emergent column correction). When the compound is in the form of a salt, the melting point corresponds to that of the compound in salt form.

### PREPARATION 1

### Ethyl (1SR,7aRS,12aSR,12bSR)-9-chloro-1,2,3,4,6,7,7a,12,12a,12b-decahydroindolo[2,3-a]quinolizine-1-carboxylate

### Step A: 1-[2-(5-Chloro-1H-indol-3-yl)ethyl]piperidin-2-one

To a solution of 100 g of 5-chlorotryptamine hydrochloride in 1.4 litres of 2-methoxyethanol there, are added 60 g of Na₂CO₂. The reaction mixture is stirred at reflux under nitrogen. A solution of 111.2 g of 5-bromovalerate in 200 ml of 2-methoxyethanol is added dropwise over a period of 5-6 hours and the mixture is heated at reflux for 24 hours. After cooling, the reaction mixture is filtered over Celite and the filtrates are concentrated under reduced pressure. The oil is extracted with 500 ml of CH₂Cl₂ and 300 ml of water. The organic phases are washed with saturated sodium chloride solution, then dried over Na₂SO₄ and concentrated under reduced pressure. The solid is recrystallised from a 9/1 acetone/pentane mixture to yield 107 g of the expected product.

Melting point: 155° C.

Mass spectrometry (EI, m/z): 276.8 (M⁺).

### Step B: 9-Chloro-2,3,4,6,7,12-hexahydro-1H-indolo[2,3-a]quinolizin-5-ylium tetrafluoroborate

To a solution of the compound of Step A above in 1.2 litres of toluene there are added 83 ml of POCl₃. The reaction mixture is heated at 90° C. for 5 hours under nitrogen. After cooling whilst stirring the mixture, 3 to 4 litres of water are added and then allowed to separate. 436 ml of a solution of HBF₄ are added dropwise to the aqueous phase. 98 g of the expected product are obtained after filtration, washing with water and then drying over P₂O₅ under.

Melting point: >280° C.

Mass spectrometry (EI, m/z): 346.5 (M⁺).

### Step C: 9-Chloro-2,3,4,6,7,12-hexahydroindolo[2,3-a]quinolizine

61 g of the product of Step B above are dissolved in 510 ml of methanol and 115 ml of deionised water. The reaction mixture is stirred vigorously and heated at reflux for 2.5 hours until dissolution occurs. Refluxing is stopped and 115 ml of 4M NaOH solution are added dropwise. After the addition is complete, the reaction mixture is cooled to 0-5° C., and 35 ml of 4M NaOH solution are added with vigorous stirring for 0.5 hour. The solid residue is filtered off, washed with water and dried over P₂O₅ in vacuo to yield 42 g of the expected product.

Melting point: 114° C.

Mass spectrometry (EI, m/z): 257.78 (M⁺).

### Step D: Ethyl (1RS)-9-chloro-2,3,4,6,7,12-hexahydroindolo[2,3-a]quinolizine-1-carboxylate

To a solution of 25 g of the compound of Step C above in 500 ml of distilled CH₂Cl₂ there are added 1.75 g of 4-(dimethylamino)pyridine and 25 ml of DIEA under an argon atmosphere. The batch is stirred at ambient temperature until dissolution occurs. A solution of 19 ml of ClCO₂Et (97%) in distilled dichloromethane is added. After stirring at ambient temperature for 12 hours, the reaction mixture is filtered, the insoluble material is rinsed with 120 ml of CH₂Cl₂ and the filtrate is concentrated under reduced pressure. Chromatography over silica gel (CH₂Cl₂) followed by recrystallisation from a 9/1 acetone/pentane mixture makes it possible to obtain 22 g of the expected product.

Melting point: 128-130° C.

Mass spectrometry (EI, m/z): 330.82 (M⁺).

### Step E: Ethyl (1SR,12bRS)-9-chloro-1,2,3,4,6,7,12,12b-octahydroindolo[2,3-a]quinolizine-1-carboxylate (cis diastereoisomer)

16 ml of acetic acid are added to a solution of 16 g of the product of Step D above in 300 ml of distilled THF. 4.4 g of NaBH₃CN are added in small portions under a nitrogen atmosphere and at 0° C.; the reaction mixture is then stirred vigorously at ambient temperature for 12 hours. Saturated Na₂CO₃ solution is then added at 0° C.; the solvent is then evaporated off under reduced pressure. 200 ml of CH₂Cl₂ and 80 ml of water are added to the residue. After extraction with CH₂Cl₂, the organic phases are washed with saturated sodium chloride solution, dried over Na₂SO₄ and then concentrated under reduced pressure. 20 ml of 2M HCl solution are added to the crude reaction mixture in 350 ml of ethanol and the reaction mixture is heated at reflux for 5 hours. The reaction mixture is concentrated under reduced pressure and the solid is dissolved in 270 ml of CH₂Cl₂. This organic solution is added to 130 ml of water. The solution is made alkaline (pH=8-9) by adding saturated Na₂CO₃ solution and is extracted with CH₂Cl₂. The organic phases are combined, washed with saturated sodium chloride solution, dried over Na₂SO₄ and then concentrated under reduced pressure. Recrystallisation from a 9/1 acetone/pentane mixture makes it possible to obtain 15 g of the expected product.

Melting point: 184° C.

Elemental analysis:

| | C % | H % | N % |
|---|---|---|---|
| Calculated: | 64.95 | 6.36 | 8.41 |
| Found: | 65.01 | 6.39 | 8.36 |

### Step F: Ethyl (1SR,12bSR)-9-chloro-1,2,3,4,6,7,12,12b-octahydroindolo[2,3-a]-quinolizine-1-carboxylate (trans diastereoisomer)

Under a flow of argon and at 0° C., 2.5 g of NaH are added in small portions to a solution of 9 g of the compound of Step E above in 300 ml of DME. After stirring at ambient temperature for 12 hours, the reaction mixture is cautiously poured onto ice and stirred for 1 hour. The organic solvent is evaporated off and the aqueous phase is cooled to 0-5° C., at which temperature 4M HCl solution is added until the pH=2-4. After stirring, saturated Na₂CO₃ solution is added until the pH=9. The reaction mixture is extracted with AcOEt and the organic phases are dried over Na₂SO₄, filtered and then concentrated under reduced pressure. The crude reaction mixture is recrystallised from a minimum of AcOEt and the solid is dried in vacuo to yield 8.3 g of the expected product.

Melting point: 88-90° C.

Infrared (v_{cm-1}): 3442; 2933; 1709

### Step G: Ethyl (1SR,7aRS,12aSR,12bSR)-9-chloro-1,2,3,4,6,7,7a,12,12a,12b-decahydroindolo[2,3-a]quinolizine-1-carboxylate (trans diastereoisomer)

To a solution of 350 ml of TFA at 0° C. under a large flow of argon there are added, alternately and in small portions, 10.3 g of the product of Step F above and 15 g of NaBH₃CN. The reaction mixture is stirred for 10 minutes at ambient temperature between each addition. After 8 hours, 3 g of NaBH₃CN are added again and then stirred for a further 12 hours at ambient temperature. 20 ml of water are added dropwise to the reaction mixture at 0° C. followed by CH₂Cl₂ until dissolution occurs. After stirring at ambient temperature, the solvents (TFA, CH₂Cl₂) are evaporated off. The aqueous phase is cooled to 0° C. and 4M NaOH solution is added. After extracting with Et₂O, the organic phases are dried over Na₂SO₄, filtered and then concentrated under reduced pressure. Recrystallisation from Et₂O followed by chromatography on silica gel (CH₂Cl₂/MeOH: 100/5) makes it possible to obtain 8 g of the expected product.

Melting point: 126-129° C.

Mass spectrometry (EI, m/z): 335.2 [M+H]³⁰

Infrared (v_{cm-1}): 3374; 2948; 1726

### EXAMPLE 1

### (5aRS,12aSR,12bSR,12cSR)-7-Chloro-2,3,4,5,5a,10,11,12a,12b,12c-decahydro-1H,12H-3a,9b,11-triazabenzo[a]naphtho[2,1,8-cde]azulen-12-one

### Step A: (1SR,7aRS,12aSR,12bSR)-9-Chloro-1,2,3,4,6,7,7a,12,12a,12b-decahydro-indolo[2,3-a]quinolizine-1-carboxamide

To a suspension, previously cooled to 0° C., of 2.77 g of ammonium chloride in 55 ml of anhydrous toluene there is added a solution of 26 ml of AlMe₃ (2M solution). The reaction mixture is stirred under nitrogen for 1 hour 30 minutes, whilst allowing the temperature to return slowly to ambient temperature. A solution of 2.89 g of the compound of Preparation 1 in 20 ml of anhydrous toluene is then added. The reaction mixture is stirred at ambient temperature for 24 hours. After cooling to 0° C., 100 ml of 1M HCl solution are added (slow addition to begin with) followed by 250 ml of water. The mixture is extracted with diethyl ether (3×100 ml). The aqueous phase is made alkaline using saturated Na₂CO₃ solution until the pH is 10 and is extracted with dichloromethane (3×100 ml). The organic phase is dried over sodium sulphate, filtered and concentrated under reduced pressure. The amide is purified by washing with diethyl ether (100 ml) to yield 2.37 g of the expected product.

Melting point: 270° C.

Elemental analysis:

| | C % | H % | N % |
|---|---|---|---|
| Calculated: | 62.84 | 6.59 | 13.74 |
| Found: | 62.59 | 6.66 | 13.71 |

### Step B: (5aRS,12aSR,12bSR,12cSR)-7-Chloro-2,3,4,5,5a,10,11,12a,12b,12c-decahydro-1H,12H-3a,9b,11-triazabenzo[a]naphtho[2,1,8-cde]azulen-12-one

To a solution of 358 mg of the compound of Step A above in 30 ml of methanol there are added 536 μl of acetic acid and 6.5 g of paraformaldehyde. The mixture is heated at reflux under nitrogen for 48 hours. After removing the solvent by evaporating in vacuo, an HCl solution (1M, 10 ml) is added to the residue and the aqueous phase is extracted with dichloromethane (3×20 ml). The aqueous phase is made alkaline to pH 9 using Na₂CO₃ and is extracted with dichloromethane (3×20 ml). The organic phase is dried over Na₂SO₄, filtered and evaporated in vacuo to yield 406 mg of a solid residue which is taken up in dichloromethane (30 ml) and NaOH solution (1M, 10 ml). The mixture is stirred at ambient temperature for 6 hours. After customary treatment, 324 mg of the expected product are obtained by recrystallisation from 35 ml of ethanol.

Melting point: >250° C.

Mass spectrometry (EI, m/z): 317 and 319.

Elemental analysis:

| | C % | H % | N % |
|---|---|---|---|
| Calculated: | 64.25 | 6.34 | 13.22 |
| Found: | 64.01 | 6.42 | 13.13 |

### EXAMPLE 2

### (5aRS,12aSR,12bSR,12cSR)-7-Chloro-2,3,5a,12a,12b,12c-hexahydro-1H,4H-3a,9b,1′-triazabenzo[a]naphtho[2,1,8-cde]azulene-10,12(5H,11H)-dione

### Step A: Ethyl (1SR,7aRS,12aSR,12bSR)-12-(aminocarbonyl)-9-chloro-1,2,3,4,6,7,7a,12,12a,-12b-decahydroindolo[2,3-a]quinolizine-1-carboxylate

A solution of 385 mg of the compound of Preparation 1 in 6 ml of anhydrous THF is added, all at once and using a syringe, to a solution of 2.33 g of KOCN and 4.43 ml of trifluoroacetic acid in 12 ml of anhydrous THF. The reaction mixture is stirred at ambient temperature under nitrogen for 2 hours. After removing the solvent in vacuo, dichloromethane (30 ml) is added to the residue. The organic phase is extracted with HCl solution (1M, 6×10 ml). The aqueous phase is adjusted to pH 9 using Na₂CO₃ and is extracted with dichloromethane (3×20 ml). The organic phase is dried over Na₂SO₄, filtered and evaporated in vacuo to yield 500 mg of the expected product. The product is used immediately in the next Step.

### Step B: (5aRS,12aSR,12bSR,12cSR)-7-Chloro-2,3,5a,12a,12b,12c-hexahydro-1H,4H-3a,9b,11-triazabenzo[a]naphtho[2,1,8-cde]azulene-10,12(5H,11H)-dione

A solution of 500 mg of the product of Step A above in 25 ml of ethanol is treated with 6.36 mg of K₂CO₃. The reaction mixture is heated at reflux for 1 h. After removal of the solvent in vacuo, water (50 ml) is added and extraction with dichloromethane (3×20 ml) is carried out. The organic phase is dried over Na₂SO₄, filtered and evaporated in vacuo. Crystallisation from ethanol (50 ml) makes it possible to obtain 220 mg of the expected product.

Melting point: 243° C.

Mass spectrometry (EI, m/z): 331.

Elemental analysis:

| | C % | H % | N % |
|---|---|---|---|
| Calculated: | 61.54 | 5.47 | 12.66 |
| Found: | 61.39 | 5.54 | 12.58 |

### EXAMPLE 3

### (12aRS,12bRS)-7-Chloro-2,3,12a,12b-tetrahydro-4H,4H-3a,9b,11-triazabenzo[a]naphtho[2,1,8-cde]azulene-10,12(5H,11H)-dione

To a solution of 201 mg of the compound of Example 2 in 5 ml of DMF (5 ml) there are added 526 mg of MnO₂. The reaction mixture is stirred at ambient temperature for 52 hours and is then filtered over Celite. The solvent is removed by evaporation in vacuo, and crystallisation from a minimum of ethanol makes it possible to obtain 80 mg of the expected product.

Melting point: 214° C.

Mass spectrometry (EI, m/z): 329.

Elemental analysis:

| | C % | H % | N % |
|---|---|---|---|
| Calculated: | 61.91 | 4.89 | 12.74 |
| Found: | 61.59 | 4.81 | 12.71 |

### EXAMPLE 4

### (5aRS,12aSR,12bSR,12cSR)-2,3,5a,12a,12b,12c-Hexahydro-1H,4H-3a,9b,11-triazabenzo[a]naphtho[2,1,8-cde]azulene-10,12(5H,11H)-dione

To a solution of 109 mg of the compound of Example 2 in 10 ml of anhydrous THF to which 140 μl of triethylamine has been added there is added a spatula “tip” of 10% palladium-on-carbon. The reactor is purged by means of several vacuum/nitrogen cycles and then the reaction mixture is placed under a hydrogen atmosphere and stirred at ambient temperature for 24 hours. The mixture is filtered over Celite and the solvent is removed by evaporation in vacuo. HCl solution (1M, 30 ml) is added to the residue; the mixture is then extracted with dichloromethane (3×20 ml). The aqueous phase is brought to pH 9 using Na₂CO₃ and is extracted with dichloromethane (3×20 ml). The organic phase is dried over Na₂SO₄, filtered and evaporated in vacuo. Crystallisation from a minimum of ethanol makes it possible to obtain 65 mg of the expected product.

Melting point: 255° C.

Mass spectrometry (EI, m/z): 298.

Elemental analysis:

| | C % | H % | N % |
|---|---|---|---|
| Calculated: | 68.67 | 6.44 | 14.13 |
| Found: | 68.56 | 6.53 | 14.10 |

### EXAMPLE 5

### (5aS,12aR,12bR,12cR)— or (5aR,12aS,12bS,12cS)-2,3,5a,12a,12b,12c-Hexahydro-1H,4H-3a,9b,11-triazabenzo[a]naphtho[2,1,8-cde]azulene-10,12(5H,11H)-dione (enantiomer α)

The compound of Example 4 is resolved by fractional crystallisation of the diastereoisomeric salts prepared by adding to a methanolic solution of the compound of Example 4 either a solution of (−)-di-O,O′-para-toluoyl-L-tartaric acid or a solution of (+)-di-O,O′-para-toluoyl-D-tartaric acid. After separation of the diastereoisomeric salt, the base is isolated by customary treatment.

Melting point: 250° C.

Optical rotation α_{D}=+95° (c=1 in CHCl₃).

Elemental analysis:

| | C % | H % | N % |
|---|---|---|---|
| Calculated: | 68.15 | 6.48 | 14.03 |
| Found: | 68.12 | 6.62 | 14.01 |

### EXAMPLE 6

### (5aS,12aR,12bR,12cR)— or (5aR,12aS,12bS,12cS)-2,3,5a,12a,12b,12c-Hexahydro-1H,4H-3a,9b,11-triazabenzo[a]naphtho[2,1,8-cde]azulene-10,12(5H,11H)-dione (enantiomer β)

The compound of Example 4 is resolved by fractional crystallisation of the diastereoisomeric salts prepared by adding to a methanolic solution of the compound of Example 4 either a solution of (−)-di-O,O′-para-toluoyl-L-tartaric acid or a solution of (+)-di-O,O′-para-toluoyl-D-tartaric acid. After separation of the diastereoisomeric salt, the base is isolated by customary treatment.

Melting point: 250° C.

Optical rotation α_{D}=−95° (c=1 in CHCl₃).

Elemental analysis:

| | C % | H % | N % |
|---|---|---|---|
| Calculated: | 68.15 | 6.48 | 14.03 |
| Found: | 68.12 | 6.57 | 13.98 |

### EXAMPLE 7

### (12aRS,12bRS)-2,3,12a,12b-Tetrahydro-1H,4H-3a,9b,11-triazabenzo[a]naphtho[2,1,8-cde]azulene-10,12(5H,11H)-dione

To a solution of 1.047 g of the compound of Example 4 in 10 ml of DMF there are added 4.3 g of MnO₂. The mixture is stirred at ambient temperature for 24 hours and is then filtered over Celite. After evaporating off the solvent in vacuo, the residue is taken up in saturated Na₂CO₃ solution (50 ml), and then water is added (300 ml). The aqueous phase is then extracted with dichloromethane (3×50 ml). The organic phase is dried over Na₂SO₄, filtered and evaporated in vacuo. Chromatography on a silica column (AcOEt/cyclohexane: 25:75) followed by recrystallisation from ethanol (75 ml) makes it possible to obtain 154 mg of the expected product.

Melting point: 235° C.

Mass spectrometry (EI, m/z): 295.

Elemental analysis:

| | C % | H % | N % |
|---|---|---|---|
| Calculated: | 69.14 | 5.80 | 14.23 |
| Found: | 69.09 | 5.77 | 14.21. |

### Pharmacological Study of Compounds of the Invention

### Example A

### Induction of Tyrosine Hydroxylase

An investigation is made, among the compounds, for the capability of bringing about an increase in the tyrosine hydroxylase (TH) protein in the locus coeruleus (LC) of the brain of the Balb/C mouse.

The animals used are male mice of the pure Balb/C strain (Charles River Laboratories) aged 7 to 8 weeks at the time of treatment.

The mice are given a single injection, by the intraperitoneal route, of the compound under test, dissolved in 0.04M HCl solution (corresponding control: 0.004M HCl), if the compound is sufficiently soluble, or in olive oil 90%/DMSO 10% (corresponding control: olive oil 90%/DMSO 10%) for compounds that are insoluble in an aqueous medium.

Three days after the injection of each compound, all the animals are sacrificed by decapitation. The extracted brains are then frozen in liquid nitrogen and stored at −80° C.

Coronal sections 8 microns thick are then taken along the posterior-anterior axis of the LC and fixed. The sections are transferred onto Immobilon-P membranes. The TH is measured by immunodetection and image analysis.

### Results:

The results for TH induction in the LC are given in Table I below.

TABLE I
Measurement of the amount of TH in the various LC sections,
numbered from 1 to 8 in the anterior-to-posterior direction,
after i.p. administration (20 mg/kg)
The results are expressed in %, relative to
the mean value of the control group¹

%

| | 1 | 2 | 3 | 4 | 5 | 6 | 7 | 8 |
|---|---|---|---|---|---|---|---|---|
| Ex. 2 | 53 | 54 | 46 | 35 | 25 | 14 | 5 | 3 |
| Ex. 3 | 55 | 58 | 47 | 48 | 33 | 19 | 5 | 3 |
| Ex. 4 | 88 | 78 | 70 | 58 | 27 | 14 | 9 | 3 |
| Ex. 7 | 63 | 67 | 48 | 47 | 43 | 23 | 7 | 6 |

¹animals treated with the same carrier

### Example B

### Affinity for Receptors

The affinity for receptors is determined according to customary methods relating the specific ligand and the receptor, which may be of animal origin or a human recombinant. The affinity was determined by the method of displacement of the labelled specific ligand by the compound under test and expressed by the dissociation constant Kᵢ.

The receptor affinity was accordingly studied for 28 conventional receptors. The study shows that the TH induction observed does not proceed by way of affinity for receptors customarily affected by psychotropic compounds, such as alpha adrenergic receptors (type α₂), 5HT receptors (type 5HT2A) or dopaminergic receptors (types D₁ et D₂).

Some compounds exhibit an affinity for sigma (σ) receptors (ligand: haloperidol) or muscarinic (M) receptors that is not insignificant.

TABLE II

| | α₂ | | 5HT2A | | D₁ | | D₂ | | M | | σ | |
|---|---|---|---|---|---|---|---|---|---|---|---|---|
| | % inhibition concentration (M) | | | | | | | | | | | |
| | 10⁻⁷ | 10⁻⁵ | 10⁻⁷ | 10⁻⁵ | 10⁻⁷ | 10⁻⁵ | 10⁻⁷ | 10⁻⁵ | 10⁻⁷ | 10⁻⁵ | 10⁻⁷ | 10⁻⁵ |
| Example 5 | — | — | — | — | — | — | — | — | — | — | — | — |
| Example 6 | — | — | — | — | — | — | — | — | — | — | — | — |
| Example 7 | — | — | 34 | 96 | — | — | — | — | — | — | — | — |

### Example C

### Predicted Metabolic Stability

The predicted metabolic stability is tested by incubation of the compounds at a concentration of 10⁻⁷M in the presence of mouse, rat or human hepatic microsomes (0.33 mg of prot/ml). After addition of NADPH (nicotinamide adenine dinucleotide phosphate, reduced form), samples are taken at 0, 5, 15, 30 and 60 minutes. The enzymatic reaction is stopped using methanol (V/V). The protein is precipitated by centrifugation and the supernatant is analysed by LC-MS-MS.

Good metabolic stability of the compounds makes it possible to envisage treatment per os.

TABLE III
% metabolic stability predicted using hepatic microsomes

| | Mouse | Rat | Human |
|---|---|---|---|
| Example 5 | 100 | 90 | 75 |
| Example 6 | 100 | 76 | 71 |

### Example E

### Pharmaceutical Composition

Formula for the preparation of 1000 tablets each containing 10 mg of active ingredient

| | |
|---|---|
| Compound of Example 7 | 10 g |
| Hydroxypropylcellulose | 2 g |
| Wheat starch | 10 g |
| Lactose | 100 g |
| Magnesium stearate | 3 g |
| Talc | 3 g |

1-12. (canceled)

13. A compound selected from those of formula (I): 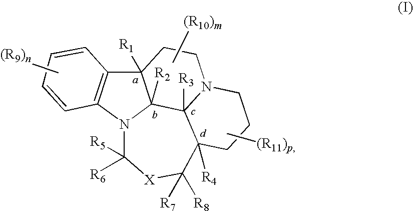
wherein:
R₁ represents a hydrogen atom, a linear or branched (C₁-C₆)alkyl group, a linear or branched (C₁-C₆)aminoalkyl group, or a linear or branched (C₁-C₆)hydroxyalkyl group;
R₂ represents a hydrogen atom;
or R₁ and R₂, together with the carbon atoms carrying them, form a carbon-carbon bond;
R₃ represents a hydrogen atom;
R₄ represents a hydrogen atom; a methyl or linear or branched (C₃-C₆)alkyl group; a linear or branched (C₁-C₆)aminoalkyl group; a linear or branched (C₁-C₆)hydroxyalkyl group; an aryl-(C₁-C₆)alkyl group in which the alkyl moiety is linear or branched; or a heterocycloalkyl-(C₁-C₆)alkyl group in which the alkyl moiety is linear or branched;
or R₃ and R₄, together with the carbon atoms carrying them, form a carbon-carbon bond;
R₅, R₆, R₇ and R₈, which may be identical or different, represent, each independently of the others, a hydrogen atom
or a pair of geminal substituents (R₅ and R₆ and/or R₇ and R₈) form an oxo, thioxo or imino group;
R₉ represents a hydrogen atom, a halogen atom, an optionally substituted, linear or branched (C₁-C₆)alkyl group, a linear or branched (C₁-C₆)alkoxy group, a hydroxy group, a cyano group, a nitro group, a linear or branched (C₁-C₆)polyhaloalkyl group, or an amino group (optionally substituted by one or two linear or branched (C₁-C₆)alkyl(s), linear or branched (C₂-C₆)alkenyl(s), it being possible for the alkyls and alkenyls to be identical or different);
R₁₀ and R₁₁, which may be identical or different, represent, each independently of the other, a hydrogen atom, a halogen atom, a linear or branched (C₁-C₆)alkyl group; a linear or branched (C₁-C₆)alkoxy group; a hydroxy group; a cyano group; a nitro group; a linear or branched (C₁-C₆)polyhaloalkyl group; or an amino group (optionally substituted by one or two linear or branched (C₁-C₆)alkyl(s), linear or branched (C₂-C₆)alkenyl(s), it being possible for the alkyls and alkenyls to be identical or different);
n represents an integer between 0 and 4 inclusive;
m represents an integer between 0 and 2 inclusive;
p represents an integer between 0 and 3 inclusive;
X represents NR₁₂,
R₁₂ represents a hydrogen atom, an optionally substituted, linear or branched (C₁-C₆)alkyl group, an optionally substituted, linear or branched (C₂-C₆)alkenyl group, an aryl-(C₁-C₆)alkyl group in which the alkyl moiety is linear or branched, or a linear or branched (C₁-C₆)polyhaloalkyl group;
its enantiomers, diastereoisomers and N-oxides, and addition salts thereof with a pharmaceutically acceptable acid or base,
it being understood that:
arylalkyl means an aryl-alkyl group in which the alkyl group denotes a linear or branched chain of 1 to 6 carbon atoms and the aryl group denotes an optionally substituted phenyl or naphthyl group;
the expression “optionally substituted” when referring to linear or branched (C₁-C₆)alkyl, linear or branched (C₂-C₆)alkenyl or arylalkyl groups means that these groups may be substituted by one or more halogen atoms, and/or by one or more groups selected from hydroxy, linear or branched (C₁-C₆)alkoxy and amino (optionally substituted by one or two identical or different, linear or branched (C₁-C₆)alkyl groups); and
a, b, c and d denote the chiral centres which may be present in the compounds of formula (I).

14. The compound of claim 13, wherein X represents NR₁₂ wherein R₁₂ represents a hydrogen atom.

15. The compound of claim 13, wherein R₁, R₂, R₃ and R₄ each represent a hydrogen atom.

16. The compound of claim 13, wherein R₁ and R₂, together with the carbon atoms carrying them, form a carbon-carbon bond.

17. The compound of claim 13, wherein R₉ represents a hydrogen or halogen atom.

18. The compound of claim 13, wherein R₁₀ and R₁₁ each represent a hydrogen atom.

19. The compound of claim 13, wherein each pair of geminal substituents (R₅ and R₆, and R₇ and R₈) form an oxo group.

20. The compound of claim 13, wherein R₅ and R₆ each represent a hydrogen atom and R₇ and R₈ form an oxo group.

21. The compound of claim 13 which is selected from:
(5aRS,12aSR,12bSR,12cSR)-7-chloro-2,3,4,5,5a,10,11,12a,12b,12c-decahydro-1H,12H-3a,9b,11-triazabenzonaphtho[2,1,8-cde]azulen-12-one,
(5aRS,12aSR,12bSR,12cSR)-7-chloro-2,3,5a,12a,12b,12c-hexahydro-1H,4H-3a,9b, 11-triazabenzonaphtho[2,1,8-cde]azulene-10,12(5H,11H)-dione,
(12aRS,12bRS)-7-chloro-2,3,12a,12b-tetrahydro-1H,4H-3a,9b,11-triazabenzonaphtho[2,1,8-cde]azulene-10,12(5H,11H)-dione,
(5aRS,12aSR,12bSR,12cSR)-2,3,5a,12a,12b,12c-hexahydro-1H,4H-3a,9b, 11-triazabenzonaphtho[2,1,8-cde]azulene-10,12(5H,11H)-dione,
(5aS,12aR,12bR,12cR)— or (5aR,12aS,12bS,12cS)-2,3,5a,12a,12b,12c-hexahydro-1H,4H-3a,9b,11-triazabenzonaphtho[2,1,8-cde]azulene-10,12(5H,11H)-dione (enantiomer α),
(5aR,12aS,12bS,124- or (5aS,12aR,12bR,12cR)-2,3,5a,12a,12b,12c-hexahydro-1H,4H-3a,9b, 11-triazabenzonaphtho[2,1,8-cde]azulene-10,12(5H,11H)-dione (enantiomer β),
(12aRS,12bRS)-2,3,12a,12b-tetrahydro-1H,4H-3a,9b,11-triazabenzo[a]naphtho[2,1,8-cde]azulene-10,12(5H,11H)-dione, and
enantiomers, diastereoisomers, and addition salts thereof with a pharmaceutically acceptable acid or base.

22. A pharmaceutical composition comprising as active ingredient at least one compound of claim 13 in combination with one or more pharmaceutically acceptable, inert, non-toxic excipients or carriers.

23. A method of treating a living animal body, including a human, afflicted with a condition selected from depression, anxiety, disorders of memory associated with aging and/or neurodegenerative diseases, and stress, comprising the step of administering to the living animal body, including a human, an amount of a compound of claim 13 which is effective for treatment of the condition.

24. A method of treating a living animal body, including a human, afflicted with Parkinson's disease, comprising the step of administering to the living animal body, including a human, an amount of a compound of claim 13 which is effective for the palliative treatment of Parkinson's disease.
